(19) United States
(12) Patent Application Publication
(10) Pub. No.: US 2014/0220640 A1
(43) Pub. Date: Aug. 7, 2014

(54) METHOD FOR MANUFACTURING DETOXIFICATED LIGNOCELLULOSIC BIOMASS HYDROLYSATE WITH DECREASED OR ELIMINATED TOXICITY AND METHOD FOR MANUFACTURING ORGANIC OR AND BIOFUEL USING THE SAME

(71) Applicants: Young Soon Um, Seoul (KR); Kyung Min Lee, Seoul (KR); Ki Yeon Kim, Seoul (KR); Yun Je Kim, Seoul (KR); Byoung In Sang, Seoul (KR)

(72) Inventors: Young Soon Um, Seoul (KR); Kyung Min Lee, Seoul (KR); Ki Yeon Kim, Seoul (KR); Yun Je Kim, Seoul (KR); Byoung In Sang, Seoul (KR)

(73) Assignee: KOREA INSTITUTE OF SCIENCE AND TECHNOLOGY, Seoul (KR)

(21) Appl. No.: 13/816,355

(22) PCT Filed: May 30, 2012
(86) PCT No.: PCT/KR2012/004243

§ 371 Date: Feb. 11, 2013

(30) Foreign Application Priority Data
Sep. 23, 2011 (KR) 10-2011-0096498

(51) Int. Cl. C12P 19/14; C12P 19/02; C12P 7/16; C12P 7/36; C12P 7/14

(52) U.S. Cl. CPC C12P 19/14; C12P 7/36; C12P 7/14; C12P 7/16; C12P 19/02
USPC 435/99; 435/151; 435/162; 435/160

(57) ABSTRACT

Disclosed is a method for detoxifying a lignocellulosic biomass hydrolysate, including: preparing a hydrolysate by pretreating a lignocellulosic biomass by hydrolysis; and decreasing or removing toxicity by adding a surfactant to the hydrolysate. The detoxifying method according to the present disclosure may effectively remove toxicity of compounds derived from lignin that inhibit the growth of and fermentation by microorganisms during the pretreatment of lignocellulosic biomass. Further, production efficiency can be improved since loss of sugar can be avoided during the detoxification and additional cost can be minimized.

### TECHNICAL FIELD

The present disclosure relates to a method for detoxifying a lignocellulosic biomass hydrolysate with decreased or eliminated toxicity and a method for preparing an organic acid or a biofuel using same.

### BACKGROUND ART

Depletion of petroleum resources and high oil price have a large impact on the entire industry including chemical industry. Also, carbon dioxide emission accompanied by the use of fossil fuels and global warming caused thereby have demanded on change toward environment-friendly, sustainable, renewable energy. The new renewable energy should satisfy the requirements of technical viability, economy, environment-friendliness, etc. Developments of alternative energy sources for replacing petroleum are actively under way, including hydropower, solar energy, wind power, hydrogen, biomass, or the like. Biomass is a renewable energy source for producing biofuel, electricity, heat, etc. from plant materials and is highly esteemed for its environment-friendliness, economy and technical viability.

During pretreatment of lignocellulosic biomass by hydrolysis, phenolic compounds and non-phenolic compounds are produced. These toxic materials inhibit the growth of and fermentation by microorganisms, leading to decreased production efficiency of organic acids and alcohols.

Thus, in order to improve production yield, it is necessary to detoxify the hydrolysate before fermentation. Detoxifying methods for removing the inhibitory materials from the lignocellulosic biomass hydrolysate may be largely classified into physicochemical methods and biological methods. However, these methods cannot effectively remove the fermentation inhibiting materials and the removal efficiency varies for different fermentation inhibitors. In addition, the removal of the fermentation inhibitors by adsorption is disadvantageous in that fermentation yield decreases since sugars are removed together during the detoxifying process.

### DISCLOSURE

### Technical Problem

The present disclosure is directed to removing or decreasing toxicity of fermentation inhibitors derived from lignin that inhibit the growth of and fermentation by microorganisms during pretreatment of lignocellulosic biomass while avoiding loss of sugar, and minimizing the cost.

### Technical Solution

In one general aspect, there is provided a method for preparing a lignocellulosic biomass hydrolysate with toxicity decreased or removed, including: preparing a hydrolysate by pretreating a lignocellulosic biomass by hydrolysis; and decreasing or removing toxicity by adding a surfactant to the hydrolysate.

In an exemplary embodiment of the present disclosure, the surfactant may react with a hydrophobic moiety of a phenolic compound in the hydrolysate and form a micelle.

In an exemplary embodiment of the present disclosure, the phenolic compound may be one or more selected from a group consisting of ferulic acid, coumaric acid, benzoic acid, syringic acid, vanillic acid, vanillin, 4-hydroxybenzoic acid, 4-hydroxybenzaldehyde and syringaldehyde.

In an exemplary embodiment of the present disclosure, the surfactant may be selected from Tween 20, Tween 40, Tween 60 and Tween 80.

In an exemplary embodiment of the present disclosure, the surfactant may be added in an amount of 0.01-10 g/L based on the total volume of the hydrolysate.

In another general aspect, there is provided a method for preparing an organic acid or a biofuel, including fermenting a lignocellulosic biomass hydrolysate with toxicity decreased or removed prepared by the method described above.

In an exemplary embodiment of the present disclosure, the fermentation may be performed by adding a microorganism to the hydrolysate.

In an exemplary embodiment of the present disclosure, the microorganism may be one or more selected from a group consisting of yeast, lactic acid bacterium, Clostridium, coliform bacterium and Bacillus.

In an exemplary embodiment of the present disclosure, the microorganism may be one or more selected from a group consisting of Anaeromyxobacter sp., Alcaligenes sp., Bacteroides sp., Bacillus sp., Clostridium sp., Escherichia sp., Lactobacillus sp., Lactococcus sp., Pichia sp., Pseudomonas sp., Ralstonia sp., Rhodococcus sp., Saccharomyces sp., Streptomyces sp., Thermus sp., Thermotoga sp., Thermoanaerobacter sp. and Zymomonas sp.

In an exemplary embodiment of the present disclosure, the microorganism may be one or more selected from a group consisting of Clostridium beijerinckii, Clostridium acetobutylicum, Clostridium butyricum, Clostridium cellulolyticum, Clostridium thermocellum, Clostridium perfringens, Clostridium sporogenes, Clostridium thermohydrosulfuricum, Clostridium kluyveri, Clostridium aciditolerans, Clostridium pasteurianum, Clostridium ljungdahlii, Clostridium autoethanogenum, Clostridium formicoaceticum, Clostridium thermoaceticum, Clostridium aceticum and Clostridium tyrobutyricum.

In an exemplary embodiment of the present disclosure, the organic acid may be lactic acid, acetic acid, butyric acid or hexanoic acid.

In an exemplary embodiment of the present disclosure, the biofuel may be acetone, ethanol or butanol as non-limiting examples.

### Advantageous Effects

The detoxifying method according to the present disclosure may effectively remove toxicity of the compounds derived from lignin that inhibit the growth of and fermentation by microorganisms during pretreatment of lignocellulosic biomass. Further, production efficiency can be improved since loss of sugar can be avoided during the detoxification and additional cost can be minimized. Accordingly, organic acids or biofuels can be produced more effectively from lignocellulosic biomass.

### DESCRIPTION OF DRAWINGS

FIG. 1 shows growth of Clostridium tyrobutyricum for different phenolic compounds.

FIG. 2 shows production of butyric acid for different phenolic compounds.

FIG. 3 shows growth of Clostridium tyrobutyricum for different phenolic compounds with or without addition of a surfactant.

FIG. 4 shows production of butyric acid using Clostridium tyrobutyricum for different phenolic compounds with or without addition of a surfactant.

FIG. 5 shows toxicity of dissolved lignin as well as growth of Clostridium tyrobutyricum and production of butyric acid with or without addition of a surfactant.

FIG. 6 shows toxicity of dissolved lignin as well as growth of Clostridium acetobutylicum and production of butanol with or without addition of a surfactant.

FIG. 7 shows toxicity of dissolved lignin as well as growth of Clostridium beijerinckii and production of butanol with or without addition of a surfactant.

### BEST MODE

Hereinafter, the present disclosure is described in more detail.

Organic acids or biofuels as alternative energy source for coping with depletion of petroleum resources and global warming are prepared by fermenting the hydrolysate of lignocellulosic biomass.

Lignocellulosic biomass is generally composed of lignocelluloses which is a complex consisting of cellulose, hemicelluloses, lignin, etc, although the chemical composition and content may vary depending on whether the wood from which it is derived is coniferous or broadleaf, species of trees, age of the tress, or the like.

Cellulose is a polysaccharide mainly consisting of β-1,4-linked glucose units. Unlike amylose, a starch whose helical structure is stabilized by glucose units bound by α-1,4 linkage, cellulose has a much stronger structure physically and chemically since it consists of a stable linear chain.

Hemicellulose is a polysaccharide having a lower degree of polymerization than cellulose. It is mainly composed of the pentose xylose and can include the pentose arabinose and hexoses such as mannose, galactose, glucose, etc. Since hemicellulose has a lower degree of polymerization and less structural regularity as compared to cellulose, it is relatively easily degraded by pretreatment of biomass.

Lignin is a complex, hydrophobic and aromatic macromolecule with a huge molecular weight, consisting of methoxylated p-coumaryl alcohol, coniferyl alcohol, sinapyl alcohol, etc. With strong chemical durability, lignin is considered as the most difficult-to-be-degraded substance among naturally occurring materials.

Lignin is covalently bonded to hemicellulose and hemicellulose is linked to cellulose via hydrogen bonding. Accordingly, lignocellulose has a structure in which a linear-chain cellulose microfibril is enclosed by hemicellulose via hydrogen bonding and hemicellulose is, in turn, enclosed by lignin via covalent bonding.

The technical and economical difficulty in production of biofuel from lignocellulosic biomass originate from the relatively high content of lignin as compared to those of the starch and sugar.

Lignocellulosic biomass may comprise 33-51 wt % of cellulose, 19-34 wt % of hemicellulose, 21-32 wt % of lignin, 0-2 wt % of ash and other components. During pretreatment, the cellulose and hemicellulose components are hydrolyzed into pentoses or hexoses including glucose, galactose, mannose, rhamnose, xylose and arabinose. In addition to the sugars, non-phenolic compounds such as furan, hydroxymethylfurfural (HMF), furfural and weak acids are produced by hydrolysis. And, the lignin components are hydrolyzed into phenolic compounds such as ferulic acid, coumaric acid, benzoic acid, syringic acid, vanillic acid, vanillin, 4-hydroxybenzoic acid, 4-hydroxybenzaldehyde, syringaldehyde, etc.

Among the compounds produced from the hydrolysis of the lignocellulosic biomass, the phenolic compounds, which are fermentation inhibitors, inhibit the growth of microorganisms and decrease the production yield of organic acids or biofuels using microorganisms.

Accordingly, for effective utilization of the lignocellulosic biomass hydrolysate, the toxicity of the phenolic compounds should be decreased. The inventors of the present disclosure have found out that, when a surfactant is added to the pretreated hydrolysate of lignocellulosic biomass, the surfactant removes or decreases the toxicity by enclosing a hydrophobic moiety of the phenolic compound in the hydrolysate and thus forming micelles. No case of using a surfactant to detoxify the fermentation inhibitors derived from lignin found in the lignocellulosic biomass hydrolysate has been reported yet.

In an exemplary embodiment of the present disclosure, the surfactant may be selected from an ionic surfactant, a non-ionic surfactant, a zwitterionic surfactant, a polymeric surfactant, a phospholipid, a biologically derived surfactant, an amino acid or a derivative thereof, derivatives of the afore-described surfactants, combinations thereof and aggregates thereof. The ionic surfactant may be anionic or cationic.

A suitable anionic surfactant includes, although not being limited thereto, alkyl sulfonate, aryl sulfonate, alkyl phosphate, alkyl phosphonate, potassium laurate, sodium lauryl sulfate, sodium dodecyl sulfate, alkyl polyoxyethylene sulfate, sodium alginate, dioctyl sodium sulfosuccinate, phosphatidic acid and a salt thereof, sodium carboxymethyl cellulose, bile acid and a salt thereof, cholic acid, deoxycholic acid, glycocholic acid, taurocholic acid, glycodeoxycholic acid, calcium carboxymethyl cellulose, stearic acid and a salt thereof, calcium stearate, phosphate, sodium dodecyl sulfate, dioctyl sulfosuccinate, dialkyl ester of sodium sulfosuccinate and phospholipid.

A suitable cationic surfactant includes, although not being limited thereto, a quaternary ammonium compound, benzalkonium chloride, cetyltrimethylammonium bromide, chitosan, lauryldimethylbenzylammonium chloride, acyl carnitine hydrochloride, alkyl pyridinium halide, cetylpyridinium chloride, cationic lipid, polymethyl methacrylate trimethylammonium bromide, a sulfonium compound, polyvinylpyrrolidone-2-dimethylaminoethyl methacrylate dimethyl sulfate, hexadecyltrimethylammonium bromide, a phosphonium compound, benzyl-di(2-chloroethyl)ethylammonium bromide, coconut trimethylammonium chloride, coconut trimethylammonium bromide, coconut methyldihydroxyethylammonium chloride, coconut methyldihydroxyethylammonium bromide, decyltriethylammonium chloride, decyldimethylhydroxyethylammonium chloride, decyldimethylhydroxyethylammonium chloride bromide, C₁₂₋₁₅ dimethylhydroxyethylammonium chloride, C₁₂₋₁₅ dimethylhydroxyethylammonium chloride bromide, coconut dimethylhydroxyethylammonium chloride, coconut dimethylhydroxyethylammonium bromide, myristyltrimethylammonium methyl sulphate, lauryldimethylbenzylammonium chloride, lauryldimethylbenzylammonium bromide, lauryldimethyl(ethenoxy)₄ammonium chloride, lauryldimethyl(ethenoxy)₄ammonium bromide, N-alkyl(C₁₂₋₁₈)dimethylbenzylammonium chloride, N-alkyl(C₁₄₋₁₈)dimethyl-benzylammonium chloride, N-tetradecyldimethylbenzylammonium chloride monohydrate, dimethyldidecylammonium chloride, N-alkyl and (C₁₂₋₁₄)dimethyl-1-napthylmethylammonium chloride, trimethylammonium halide alkyl trimethylammonium salt, a dialkyl dimethylammonium salt, lauryltrimethylammonium chloride, an ethoxylated alkylamidoalkyldialkylammonium salt, an ethoxylated trialkylammonium salt, dialkylbenzene dialkylammonium chloride, N-didecyldimethylammonium chloride, N-tetradecyldimethylbenzylammonium chloride monohydrate, N-alkyl(C₁₂₋₁₄)dimethyl-1-napthylmethylammonium chloride, dodecyldimethylbenzylammonium chloride, dialkylbenzenealkylammonium chloride, lauryltrimethylammonium chloride, alkylbenzylmethylammonium chloride, alkylbenzyldimethylammonium bromide, C₁₂ trimethylammonium bromide, C₁₅ trimethylammonium bromide, C₁₇ trimethylammonium bromide, dodecylbenzyl triethylammonium chloride, polydiallyldimethylammonium chloride (poly-DADMAC), dimethylammonium chloride, alkyldimethylammonium halogenide, tricetylmethylammonium chloride, decyltrimethylammonium bromide, dodecyltriethylammonium bromide, tetradecyltrimethylammonium bromide, methyltrioctylammonium chloride, “Polyquat 10” (a mixture of polymeric quaternary ammonium compounds), tetrabutylammonium bromide, benzyltrimethylammonium bromide, choline ester, benzalkonium chloride, stearalkonium chloride, cetylpyridinium bromide, cetylpyridinium chloride, a halide salt of quaternized polyoxyethylalkylamine, “MIRAPOL” (polyquaternium-2) “Alkaquat” (alkyldimethylbenzylammonium chloride, available from Rhodia), an alkylpyridinium salt, amine, an amine salt, an imidazolinium salt, protonated quaternary acrylamide, methylated quaternary polymer, cationic guar gum, dodecyltrimethylammonium bromide, triethanolamine and poloxamine.

A suitable non-ionic surfactant includes, although not being limited thereto, polyoxyethylene fatty alcohol ether, polyoxyethylene sorbitan fatty acid ester, alkyl polyoxyethylene sulfate, polyoxyethylene fatty acid ester, sorbitan ester, glyceryl ester, glycerol monostearate, polyethylene glycol, polypropylene glycol, polypropylene glycol ester, cetyl alcohol, cetostearyl alcohol, stearyl alcohol, aryl alkyl polyether alcohol, a polyoxyethylene-polyoxypropylene copolymer, poloxamer, poloxamine, methyl cellulose, hydroxycellulose, hydroxymethyl cellulose, hydroxypropyl cellulose, hydroxypropylmethyl cellulose, noncrystalline cellulose, polysaccharide, starch, a starch derivative, hydroxyethyl starch, polyvinyl alcohol, polyvinylpyrrolidone, triethanolamine stearate, amine oxide, dextran, glycerol, acacia gum, cholesterol, tragacanth, cetostearyl alcohol, cetomacrogol emulsifying wax, polyoxyethylene alkyl ether, a polyoxyethylene castor oil derivative, polyoxyethylene stearate, hydroxyethyl cellulose, hydroxypropylmethyl cellulose phthalate, a 4-(1,1,3,3-tetramethylbutyl)phenol polymer with ethylene oxide and formaldehyde, alkyl aryl polyether sulfonate, a mixture of sucrose stearate and sucrose distearate, p-isononylphenoxypoly(glycidol), decanoyl-N-methylglucamide, n-decyl-β-D-glucopyranoside, n-decyl-β-D-maltopyranoside, n-dodecyl-β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-nonyl-β-D-glucopyranoside, octanoyl-N-methylglucamide, n-octyl-β-D-glucopyranoside, octyl-β-D-thioglucopyranoside, PEG-cholesterol, a PEG-cholesterol derivative, PEG-vitamin A, PEG-vitamin E and a random copolymer of vinyl acetate and vinyl pyrrolidone.

A zwitterionic surfactant is electrically neutral but has both localized positive and negative charges in the same molecule. A suitable zwitterionic surfactant includes, although not being limited thereto, a zwitterionic phospholipid. A suitable phospholipid includes phosphatidylcholine, phosphatidylethanolamine and diacylglycerophosphoethanolamine (e.g., dimyristoylglycerophosphoethanolamine (DMPE), dipalmitoylglycerophosphoethanolamine (DPPE), distearoylglycerophosphoethanolamine (DSPE) and dioleolylglycerophosphoethanolamine (DOPE)). In an exemplary embodiment of the present disclosure, a phospholipid mixture comprising an anionic phospholipid and a zwitterionic phospholipid may be used. Such a mixture includes, although not being limited thereto, lysophospholipid, egg or soybean phospholipid or random compositions thereof.

A suitable polymeric surfactant includes, although not being limited thereto, polyamide, polycarbonate, polyalkylene, polyalkylene glycol, polyalkylene oxide, polyalkylene terephthalate, polyvinyl alcohol, polyvinyl ether, polyvinyl ester, polyvinyl halide, polyvinylpyrrolidone, polyglycolide, polysiloxane, polyurethane and a copolymer thereof, alkyl cellulose, hydroxyalkyl cellulose, cellulose ether, cellulose ester, nitrocellulose, a polymer of acrylic and methacrylic esters, methyl cellulose, ethyl cellulose, hydroxypropyl cellulose, hydroxypropylmethyl cellulose, hydroxybutylmethyl cellulose, cellulose acetate, cellulose propionate, cellulose acetate butyrate, cellulose acetate phthalate, carboxyethyl cellulose, cellulose triacetate, a sodium salt of cellulose sulfate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl acetate), polyvinyl chloride and polystyrene.

A suitable biologically derived surfactant includes, although not being limited thereto, lipoprotein, gelatin, casein, lysozyme, albumin, heparin, hirudin or other proteins.

Specifically, a non-ionic surfactant, for example, Tween 20 (Polysorbate 20), Tween 40 (Polysorbate 40), Tween 60 (Polysorbate 60) or Tween 80 (Polysorbate 80) may be used.

The surfactant may be added in an amount of 0.01-10 g/L, specifically 0.5-5 g/L, more specifically 1 g/L, based on the volume of the hydrolysate. When the addition amount of the surfactant is less than 0.01 g/L, detoxifying effect may be only slight.

In an exemplary embodiment of the present disclosure, the lignocellulosic biomass hydrolysate comprises 50 g/L of glucose, 23 g/L of xylose and mannose and 0.67 g/L of phenolic compounds such as ferulic acid, coumaric acid, benzoic acid, syringic acid, vanillic acid, vanillin, 4-hydroxybenzoic acid, 4-hydroxybenzaldehyde, syringaldehyde, etc., which are fermentation inhibitors derived from lignin produced during the pretreatment.

Among the above-described components of the hydrolysate, the fermentation inhibitors derived from lignin inhibit the growth of microorganisms and decrease the productivity of organic acids or bio-alcohol by impairing the function of the cellular membrane of the microorganisms or breaking the electrochemical balance of the cellular membrane, and greatly influence in fermentation of organic acids or biofuels by the microorganisms.

In another aspect, the present disclosure provides a method for preparing an organic acid or a biofuel, comprising fermenting the lignocellulosic biomass hydrolysate with toxicity decreased or removed prepared by the above-described detoxifying method.

The hydrolysate comprises sugar that can be fermented by microorganisms.

The fermentation may be achieved through biological treatment of the hydrolysate using microorganisms. That is to say, the fermentation of the hydrolysate may be achieved by adding microorganisms to the hydrolysate. The microorganism used to ferment the hydrolysate may be selected considering productivity of carboxylic acid, resistance to carboxylic acid, resistance to fermentation inhibitors that may remain in the hydrolysate, fermenting ability for pentoses and hexoses, or the like.

The microorganism may be one or more selected, for example, from a group consisting of yeast, lactic acid bacterium, Clostridium, coliform bacterium and Bacillus, although not being particularly limited thereto. These microorganisms can produce carboxylic acids or their carboxylic acid producing ability may be conferred or improved through transformation.

As specific examples of the microorganism, Anaeromyxobacter sp., Alcaligenes sp., Bacteroides sp., Bacillus sp., Clostridium sp., Escherichia sp., Lactobacillus sp., Lactococcus sp., Pichia sp., Pseudomonas sp., Ralstonia sp., Rhodococcus sp., Saccharomyces sp., Streptomyces sp., Thermus sp., Thermotoga sp., Thermoanaerobacter sp., Zymomonas sp., etc. may be used alone or in combination.

Examples of the microorganism belonging to the genus Clostridium include, specifically, Clostridium beijerinckii, Clostridium acetobutylicum, Clostridium butyricum, Clostridium cellulolyticum, Clostridium thermocellum, Clostridium perfringens, Clostridium sporogenes, Clostridium thermohydrosulfuricum, Clostridium kluyveri, Clostridium aciditolerans, Clostridium pasteurianum, Clostridium ljungdahlii, Clostridium autoethanogenum, Clostridium formicaceticum, Clostridium thermoaceticum, Clostridium aceticum and Clostridium tyrobutyricum and they may be used alone or in combination.

The produced organic acid or biofuel may be different depending on the microorganism. As non-limiting examples, the organic acid may be lactic acid, acetic acid, butyric acid or hexanoic acid and the biofuel may be acetone, ethanol or butanol. The biofuel may be produced from the produced organic acid.

In the present disclosure, the toxicity of phenolic compounds, which are major inhibitors of butyric acid fermentation from the pretreated lignocellulosic biomass hydrolysate, is decreased by adding the surfactant. This allows to avoid loss of sugar, which is the disadvantage of the existing physicochemical or biological detoxifying method.

The hydrolysate pretreated according to the present disclosure is applicable to fermentation by any microorganism capable of producing a bioalcohol, such as yeast, Clostridium, coliform bacterium, etc., and an organic acid or a biofuel may be prepared.

### MODE FOR INVENTION

Hereinafter, the present disclosure will be described in detail through examples. However, the following examples are for illustrative purposes only and it will be apparent to those of ordinary skill in the art that the scope of the present disclosure is not limited by the examples.

### Example 1

### Effect of Phenolic Compound on Growth of Microorganism and Production of Butanol or Butyric Acid

In order to investigate the effect of phenolic compounds on the growth of microorganisms and production of butanol or butyric acid by the microorganisms, microorganisms were cultured in a medium containing phenolic compounds and the cell weight of the microorganisms and the concentration of produced butanol and butyric acid was measured.

A butyric acid fermentation medium included 20 g of glucose, 5 g of yeast extract, 0.2 g of magnesium sulfate, 0.01 g of manganese sulfate, 0.01 g of iron sulfate, 0.01 g of sodium chloride, 0.5 g of monopotassium phosphate (KH₂PO₄), 0.5 g of dipotassium phosphate (K₂HPO₄) and 2 g of ammonium acetate per liter. And, a butanol fermentation medium included 20 g of glucose, 5 g of yeast extract, 0.2 g of magnesium sulfate, 0.01 g of manganese sulfate, 0.01 g of iron sulfate, 0.01 g of sodium chloride, 0.5 g of monopotassium phosphate (KH₂PO₄), 0.5 g of dipotassium phosphate (K₂HPO₄) and 2 g of ammonium acetate per liter. Each medium was flushed with argon gas and sterilized at 121° C. for 15 minutes before measurement. Initial pH was adjusted to 6.8 with 1 N potassium hydroxide (KOH).

p-Coumaric acid, ferulic acid, syringaldehyde and vanillic acid, 1 g/L each, were added to the medium as phenolic compounds. A medium with no phenolic compound added was used as control.

Butyric acid fermentation was conducted using Clostridium tyrobutyricum (American Type Culture Collection, ATCC 25755) after culturing for two passages. Butanol fermentation was conducted using Clostridium acetobutylicum (ATCC 824) and Clostridium beijerinckii (National Collection of Industrial, Marine and Food Bacteria, NCIMB 8052) after culturing for two passages.

Butyric acid and butanol fermentation was carried out by adding the culture fluid to a batch fermenter. Batch culture was performed by adding 20 mL of the medium to a 60-mL serum bottle, adding the culture fluid with an amount of 5% based on the medium and then incubating in a shaking incubator at 37° C. and 150 rpm.

The concentration of phenolic compounds, furan compounds, sugars and acetic acid was analyzed by liquid chromatography (Agilent model 1200). The phenolic compounds were detected with a diode array detector using a Zorbax eclipse XDB-C18 column (150×4.6 mm, 3.5 μm). The sugars and acetic acid were detected with a refractive index detector using a Aminex HPX-87H column (300×7.8 mm).

The growth of the microorganisms was evaluated by measuring absorbance at 600 nm using a spectrophotometer (UVmini-1240, Shimadzu).

The concentration of butyric acid and butanol was analyzed using a gas chromatography system (Agilent Technologies 6890N Network GC system) equipped with a flame ionization detector. A HP-INNOWax column (30 m×250 μm×0.25 μm, Agilent Technologies) was used.

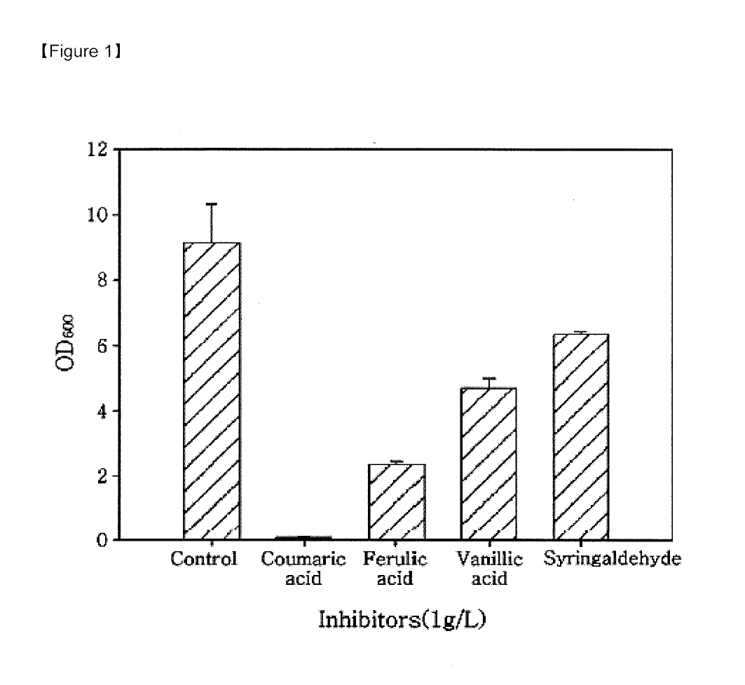

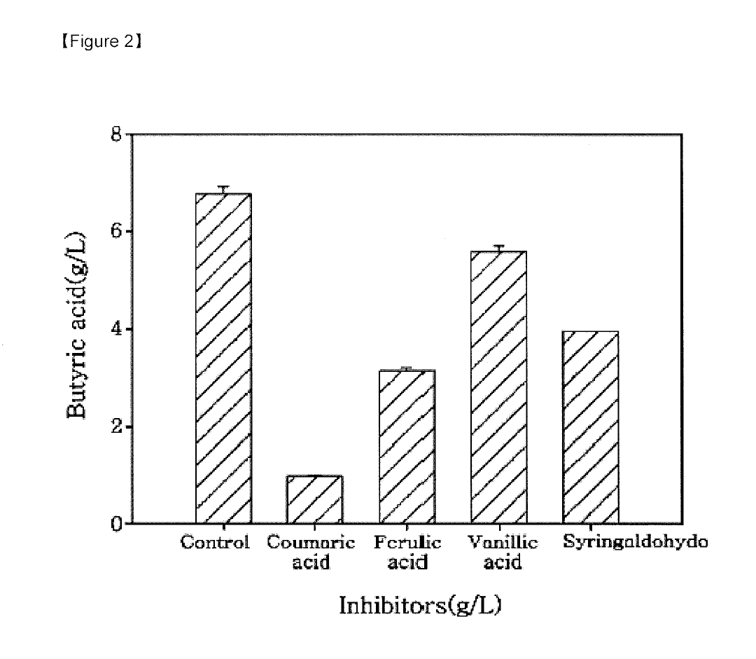

The result is shown in FIG. 1 and FIG. 2

In FIG. 1 and FIG. 2, control is the result for the case wherein no fermentation inhibitor was included. In FIG. 1, the horizontal axis represents different fermentation inhibitors and the vertical axis represents the growth of Clostridium tyrobutyricum as absorbance (optical density) measured at 600 nm.

It can be seen that toxicity increases in the order of coumaric acid, ferulic acid, vanillic acid and syringaldehyde and all the phenolic compounds inhibit the growth of Clostridium tyrobutyricum.

The result of measuring the concentration of produced butyric acid is shown in FIG. 2. From FIG. 2, it can be seen that all the phenolic compounds inhibit the production of butyric acid.

### Example 2

### Effect of Phenolic Compound and Surfactant on Growth of Microorganism and Production of Butanol or Butyric Acid

A surfactant was used to reduce inhibition of fermentation by the phenolic compounds found in the lignocellulosic biomass hydrolysate. Toxicity of each phenolic compound and water-soluble lignin was evaluated and detoxifying effect by a surfactant was measured. p-Coumaric acid, ferulic acid, syringaldehyde and vanillic acid were selected as phenolic compounds produced during pretreatment of lignocellulosic biomass for evaluation of the toxicity and detoxifying effect.

As the surfactant, Tween 80 (BioXtra, Sigma, viscous liquid) was used. The phenolic compound and Tween 80 were added at an amount of 1 g/L to the medium of Example 1. A medium with no phenolic compound or Tween 80 added was used as control.

The growth of Clostridium tyrobutyricum (ATCC 25755) and production of butyric acid thereby in the medium containing the phenolic compounds, 1 g/L each, and 1 g/L of Tween 80 were measured.

Other experimental conditions were the same as in Example 1.

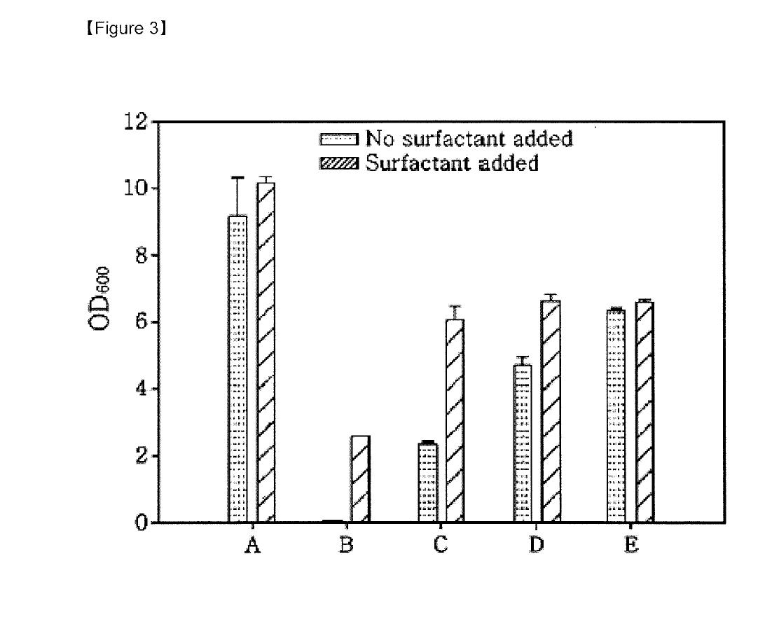

The growth of Clostridium tyrobutyricum (ATCC 25755) depending on different phenolic compounds and addition of the surfactant is shown in FIG. 3.

As seen from FIG. 3, all the tested phenolic compounds inhibit the growth of Clostridium tyrobutyricum. In FIG. 3, A is the result for the control with no phenolic compound added, B for the case with p-coumaric acid added, C for the case with ferulic acid added, D for the case with vanillic acid added and E for the case with syringaldehyde added, with or without the surfactant added. In FIG. 3, the vertical axis represents absorbance indicative of the microorganism growth.

Among the phenolic compounds p-coumaric acid (B) exhibited the highest toxicity, inhibiting the growth of the microorganism by 99%, followed by ferulic acid (C, 74%), vanillic acid (D, 48%) and syringaldehyde (E, 30%).

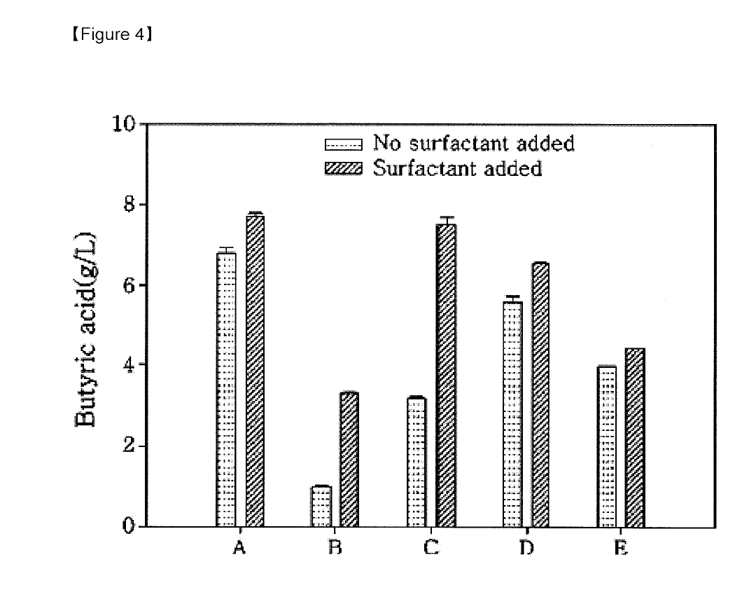

FIG. 4 shows the concentration of butyric acid produced using Clostridium tyrobutyricum (ATCC 25755) depending on different phenolic compounds and addition of the surfactant. In FIG. 4, A is the result for the control with no phenolic compound added, B for the case with p-coumaric acid added, C for the case with ferulic acid added, D for the case with vanillic acid added and E for the case with syringaldehyde added, with or without the surfactant added.

From FIG. 4, it can be seen that the production of butyric acid is inhibited by the phenolic compounds. When the surfactant was added, a higher detoxifying effect was observed for p-coumaric acid and ferulic acid, which resulted in more inhibition, than vanillic acid and syringaldehyde.

### Example 3

### Effect of Dissolved Lignin and Surfactant on Growth of Microorganism

Effect of phenolic polymer compounds or dissolved lignin (alkali, Sigma Aldrich 471003) not phenolic monomer compound, that may be contained during pretreatment, and addition of a surfactant on the growth of Clostridium tyrobutyricum, Clostridium acetobutylicum and Clostridium beijerinckii was investigated.

1 g/L of lignin (alkali, Sigma Aldrich 471003) was added to the medium of Example 1 instead of the phenolic compounds. The microorganisms were cultured after adding 1 g/L of Tween 80 to the medium. As control, a medium with no lignin or Tween 80 was used.

Other experimental conditions were the same as in Example 1 or 2.

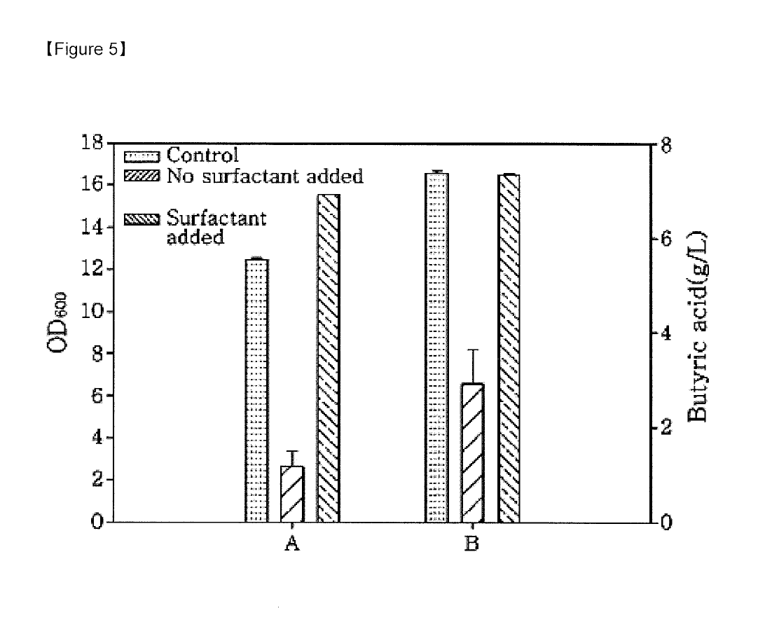

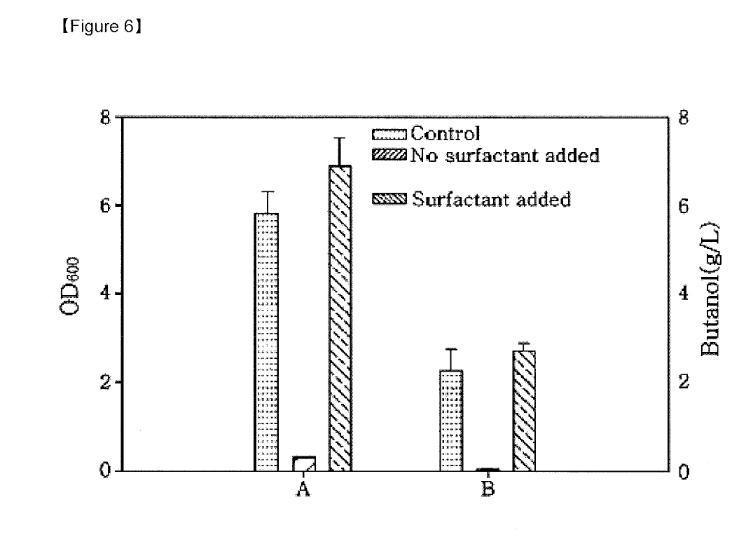

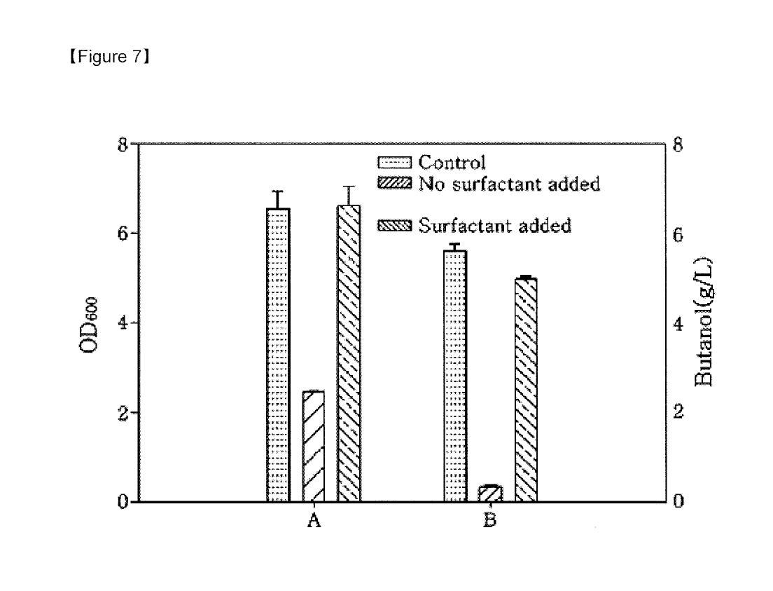

The result is shown in FIGS. 5-7. FIGS. 5-7 show the result of measuring absorbance indicative of microorganism growth and concentration of produced butyric acid or butanol for the control with no lignin added and for the case with the lignin added with or without addition of the surfactant. In FIG. 5, A is the absorbance indicative of the growth of Clostridium tyrobutyricum and B is the concentration of the produced butyric acid. In FIG. 6, A is the absorbance indicative of the growth of Clostridium acetobutylicum and B is the concentration of the produced butanol. In FIG. 7, A is the absorbance indicative of the growth of and Clostridium beijerinckii and B is the concentration of the produced butanol.

As seen from FIGS. 5-7, the dissolved lignin (alkali, Sigma Aldrich 471003) inhibits the growth of Clostridium tyrobutyricum, Clostridium acetobutylicum and Clostridium beijerinckii and the production of butyric acid and butanol thereby. It can be seen that the surfactant exerts a strong toxicity effect since the growth of the microorganisms and the production of butyric acid and butanol thereby become similar to those of the control when the surfactant is added.

While the exemplary embodiments have been shown and described, it will be understood by those skilled in the art that various changes in form and details may be made thereto without departing from the spirit and scope of this disclosure as defined by the appended claims.

### INDUSTRIAL APPLICABILITY

The detoxifying method according to the present disclosure may effectively remove toxicity of the compounds derived from lignin that inhibit the growth of and fermentation by microorganisms produced during pretreatment. Further, production efficiency can be improved since loss of sugar can be avoided during the detoxification and additional cost can be minimized. Accordingly, organic acids or biofuels can be produced more effectively from lignocellulosic biomass.

1. A method for preparing a lignocellulosic biomass hydrolysate with toxicity decreased or removed, comprising:
preparing a hydrolysate by pretreating a lignocellulosic biomass by hydrolysis; and
decreasing or removing toxicity by adding a surfactant to the hydrolysate.

2. The method for preparing a lignocellulosic biomass hydrolysate with toxicity decreased or removed according to claim 1, wherein the surfactant reacts with a hydrophobic moiety of a phenolic compound in the hydrolysate and forms micelles.

3. The method for preparing a lignocellulosic biomass hydrolysate with toxicity decreased or removed according to claim 1, wherein the phenolic compound is one or more selected from a group consisting of ferulic acid, coumaric acid, benzoic acid, syringic acid, vanillic acid, vanillin, 4-hydroxybenzoic acid, 4-hydroxybenzaldehyde and syringaldehyde.

4. The method for preparing a lignocellulosic biomass hydrolysate with toxicity decreased or removed according to claim 1, wherein the surfactant comprises one selected from Tween 20, Tween 40, Tween 60 and Tween 80.

5. The method for preparing a lignocellulosic biomass hydrolysate with toxicity decreased or removed according to claim 1, wherein the surfactant is added in an amount of 0.01-10 g/L based on the total volume of the hydrolysate.

6. A method for preparing an organic acid or a biofuel, comprising fermenting a lignocellulosic biomass hydrolysate with toxicity decreased or removed prepared by the method according to claim 1.

7. The method for preparing an organic acid or a biofuel according to claim 6, wherein the fermentation is performed by adding a microorganism to the hydrolysate.

8. The method for preparing an organic acid or a biofuel according to claim 7, wherein the microorganism is one or more selected from a group consisting of yeast, lactic acid bacterium, Clostridium, coliform bacterium and Bacillus.

9. The method for preparing an organic acid or a biofuel according to claim 7, wherein the microorganism is one or more selected from a group consisting of Anaeromyxobacter, Alcaligenes, Bacteroides, Bacillus, Clostridium, Escherichia, Lactobacillus, Lactococcus, Pichia, Pseudomonas, Ralstonia, Rhodococcus, Saccharomyces, Streptomyces, Thermus, Thermotoga, Thermoanaerobacter and Zymomonas.

10. The method for preparing an organic acid or a biofuel according to claim 7, wherein the microorganism is one or more selected from a group consisting of Clostridium beijerinckii, Clostridium acetobutylicum, Clostridium butyricum, Clostridium cellulolyticum, Clostridium thermocellum, Clostridium perfringens, Clostridium sporogenes, Clostridium thermohydrosulfuricum, Clostridium kluyveri, Clostridium aciditolerans, Clostridium pasteurianum, Clostridium ljungdahlii, Clostridium autoethanogenum, Clostridium formicaceticum, Clostridium thermoaceticum, Clostridium aceticum and Clostridium tyrobutyricum.

11. The method for preparing an organic acid or a biofuel according to claim 6, wherein the organic acid is lactic acid, acetic acid, butyric acid or hexanoic acid.

12. The method for preparing an organic acid or a biofuel according to claim 6, wherein the biofuel is acetone, ethanol or butanol.
